(19) United States
(12) Patent Application Publication
(10) Pub. No.: US 2004/0259878 A1
(43) Pub. Date: Dec. 23, 2004

(54) Fluoromethotrexates and uses therefor

(76) Inventors: Jason Koutcher, New Rochelle, NY (US); William G. Bornmann, Missouri City, TX (US)

Correspondence Address: Benjamin Aaron Adler, ADLER & ASSOCIATES, 8011 Candle Lane, Houston, TX, 77071, US

(21) Appl. No.: 10/777,839

(22) Filed: Feb. 12, 2004

Related U.S. Application Data
(60) Provisional application No. 60/530,933, filed on Dec. 19, 2003.
(60) Provisional application No. 60/447,178, filed on Feb. 13, 2003.

(51) Int. Cl. A61K 31/525; C07D 475/02

(52) U.S. Cl. 514/251; 544/260

(57) ABSTRACT

Provided herein is are novel fluorine-substituted methotrexates and methods of using magnetic resonance spectroscopy of these novel compounds to determine the sensitivity of or resistance of a tumor to methotrexate. Such methods may be used to predict the efficacy of methotrexate therapy or to devise alternate therapeutic strategies. Also provided are methods of treating tumors sensitive to methotrexate.

### CROSS-REFERENCE TO RELATED APPLICATIONS

This non-provisional applications claims benefit of provisional application U.S. Ser. No. 60/530,933, filed Feb. 19, 2003, now abandoned and of provisional application U.S. Ser. No. 60/447,178, filed Feb. 13, 2003, now abandoned.

### BACKGROUND OF THE INVENTION

1. Field of the Invention

The present invention relates generally to the fields of magnetic resonance spectroscopy, biochemistry and oncology. More specifically, the present invention provides a fluorine-labeled methotrexate, methods of treating cancers with the fluorine-labeled methotrexate and methods of using ¹⁹F magnetic resonance spectroscopy as a diagnostic for methotrexate resistance in tumors.

2. Description of the Related Art

Non-invasive in vivo monitoring of the tumor uptake and retention of anti-neoplastic agents in patients via magnetic resonance spectroscopy (MRS) is often of limited utility due to the low plasma concentrations of drug achievable without unacceptable patient toxicity (1). There are exceptions wherein previously published reports have demonstrated the in vivo MR-detection of chemotherapeutic agents administered at high doses including ¹³C-labeled temozolomide (2), iproplatin investigated via ¹H magnetic resonance spectroscopy with multiple quantum coherence transfer techniques (3) and the ³¹P-containing agents ifosfamide and cyclophosphamide (4). However, achieving acceptable signal to noise ratio in monitoring pharmacokinetics is challenging.

One case in which more robust sensitivity can be achieved is the in vivo monitoring of the uptake and metabolism of 5-fluorouracil (5-FU) via ¹⁹F magnetic resonance spectroscopy (5). In vivo monitoring of pharmacokinetics of 5-FU is possible due to the high plasma concentration of drug that is achieved clinically, the relatively high sensitivity of the 19F nucleus (83% of that of ¹H) in the magnetic resonance spectroscopy experiment and the absence of background ¹⁹F signal from endogenous metabolites.

Another antineoplastic agent routinely administered at very high dosage, achieving plasma concentrations up to 1 mM, is methotrexate. Previous reports in the literature detail the synthesis of other ¹⁹F labeled antifolates (6-7). However, in studies that compared cytotoxic efficacy of methotrexate with one of these fluorine-containing analogs in which the fluorine substitution was on the γ-glutamyl moiety of methotrexate (7) the cytotoxicity of the fluorine-containing species was found to be some 3 orders of magnitude lower. It was determined that this was the result of impaired poly-glutamylation of the fluorine-substituted species.

The current standard of care for the patient with osteosarcoma involves an initial regimen of high-dose methotrexate for up to four cycles over the course of ten weeks administered in conjunction with cisplatin and doxorubicin. However, it is known that >50% of osteosarcoma tumors in patients exhibit molecular evidence of methotrexate-resistance (8-10). Additionally, high-dose methotrexate therapy is not without complication.

Many of the clinically observed modes of resistance to methotrexate therapy involve reduced uptake and/or reduced intracellular retention and it has been postulated that tumor uptake and retention is a major indicator of the therapeutic efficacy of antineoplastic agents (10-11). A number of factors at the cellular level contribute to methotrexate resistance in human cancers (8-12). These factors can include a failure of the cancer cell to transport the drug into the intracellular space or a failure to retain the drug intracellularly. The dianionic methotrexate molecule is primarily transported into the intracellular space via the reduced folate carrier (RFC). At very high extracellular methotrexate concentrations a small diffusional contribution has also been observed in vitro (13).

The cytotoxicity of this drug is further potentiated via the action of the enzyme folylpolyglutamylate synthetase (FPGS). The folylpolyglutamylate synthetase enzyme conjugates multiple anionic glutamate residues to methotrexate increasing intracellular retention. Decreased RFC activity is a common intrinsic methotrexate-resistance mechanism in high grade osteosarcoma in humans (9) with decreased folylpolyglutamylate synthetase-activity representing a common mechanism of intrinsic resistance in soft tissue sarcoma (12). These and other means of resistance also can be acquired following initial treatment with high-dose methotrexate therapy (10-11, 14).

In the past, emphasis has been placed on monitoring plasma concentrations of MTX as a means to ensure therapeutic efficacy (15-16). However, it is fundamentally important that any cytotoxic drug be delivered to the target tissue. Imaging is one way to determine the degree of localization of a therapeutic in an individual.

In oncology, MRI is routinely used in the clinical decision making process. MR spectroscopy, on the other hand, is currently used primarily as a research tool, but is poised to play an ever-increasing role in the clinical setting (17-18). Because they are typically localized to the extremities, osteosarcomas in the clinical setting are amenable to MR interrogation via surface coil with resulting benefits in terms of sensitivity (19).

The prior art is deficient in the lack of a non-invasive real-time diagnostic tool capable of predicting therapeutic efficacy at an early stage of cancer development for managing therapy in patients. The inventors have recognized a need in the art for an improvement in the synthesis of fluorine-labeled methotrexate analogs and the use of magnetic resonance spectroscopy to assay tumor sensitivity or resistance to methotrexate. The present invention fulfills this longstanding need and desire in the art.

### SUMMARY OF THE INVENTION

The present invention is directed to a compound having the structure:

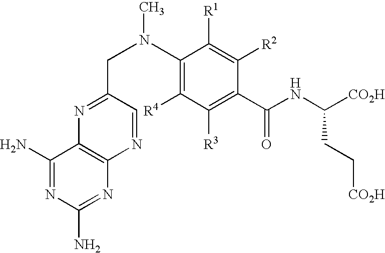

wherein R¹-R⁴ are independently fluorine or hydrogen such that at least one of R¹-R⁴ and no more than two of R¹-R⁴ are fluorine.

The present invention also is directed to a non-invasive method of monitoring tumor tissue concentration of an anticancer drug in real time. The method comprises administering an amount of the compound described supra over a period of time to an individual with the tumor. At least one time point is selected during and/or after administration of the compound and a magnetic resonance spectrum of a ¹⁹F chemical shift of the compound in a volume of the tumor at the time point(s) is acquired. The ratio of signal intensities of the ¹⁹F shift and an external standard is correlated positively with the amount of the compound in the volume of tumor at the time point(s) thereby monitoring the tumor tissue concentration of the compound in real time.

The present invention is directed further to a method of non-invasively categorizing in real time whether a tumor as sensitive to or resistant to methotrexate. The method comprises administering an amount of the compound described supra over a period of time to an individual with the tumor and acquiring a magnetic resonance spectrum of an ¹⁹F chemical shift of the compound in a volume of the tumor at a time point selected after the end of the period of administration. The ratio of signal intensities of the ¹⁹F shift and an external standard is correlated positively with concentration of the compound in the volume of tumor at the time point, where a high concentration of the compound relative to the amount administered indicates the tumor is sensitive to the compound or where a low concentration of the compound relative to the amount administered indicates the tumor is resistant to the compound, thereby categorizing said tumor in real time.

The present invention is directed further to an alternate method of non-invasively categorizing a tumor as sensitive to or resistant to methotrexate in real time. The method comprises acquiring magnetic resonance spectra of an ¹⁹F chemical shift of the compound described supra in a volume of the tumor at a first time point selected near the middle of the period of administration and at a second time point selected after the end of the period of administration. The ratio of signal intensities of the ¹⁹F shift and an external standard is positively correlated with concentration of the compound in the volume of tumor at the time points. The concentrations of the compound in the tumor volume at the first and second time points are compared, where an increase in concentration from the first time point to the second time point indicates the tumor is sensitive to the compound or, alternatively, a decrease or no increase in concentration from the first time point to the second point indicates the tumor is resistant to the compound, thereby categorizing the tumor in real time.

The present invention is directed further still to a method of treating a cancer sensitive to methotrexate in an individual comprising administering a therapeutic amount of the compound described supra over a continuous period of time at least once to the individual to reduce tumor burden of the cancer thereby treating the cancer.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 depicts a synthetic scheme for 3′-fluoromethotrexate (FMTX).

FIG. 2 depicts an alternate synthetic scheme for intermediate 6.

FIGS. 3A-3C depict energy-minimized structures based on molecular modeling. FIG. 3A: Schematic representation of fluoro-methotrexate bound to thymidylate synthase. FIG. 3B: Schematic representation of methotrexate xray structure (green), methotrexate minimized (yellow) and fluoro-methotrexate (red) bound to thymidylate synthase. FIG. 3C: M. tuberculosis DHFR-MTX (methotrexate in yellow) and 3′-fluoromethotrexate (red) energy minimized complexes with the overlayed X-ray structure (methotrexate in green) of the complex.

FIG. 4 depicts the in vitro dose response curves for the antifolates methotrexate and the 3′fluoro-analog, FMTX, against the human fibrosarcoma cell line HT-1080 for a 24 hour exposure. Resulting IC₅₀ values are reported in Table 1.

FIG. 5 demonstrates that inversion recovery data fit to equation 1 resulted in an estimated T₁ value of 0.60 sec (r=0.99) for FMTX in a blood plasma phantom sample at 4.7 T and 37° C.

FIG. 6 depicts the serial, 9-minute 19F MR spectra showing in vivo drug uptake of FMTX in a human LNCaP prostate tumor xenograft grown on the flank of a nude mouse. For display purposes the raw FID data is multiplied by a 30 Hz exponential, matched filtering. MR acquisition parameters are described in Example 8.

FIG. 7 shows 3′-fluoromethotrexate plasma pharmacokinetics.

FIG. 8 shows 3′-fluoromethotrexate tumor tissue pharmacokinetics as measured via ¹⁹F MR. For clarity error bars, ±sem, are only shown for the last 3 timepoints.

FIG. 9 HT-1080 control and post-treatment tumor regrowth following an i.v. bolus of 400 mg/kg 3′-fluoromethotrexate. Therapy growth curves which are normalized to tumor volume, Vᵢₙᵢₜ=1.

FIG. 10 depicts the tumor therapeutic response as a function of AUC₂₂₅₋₂₇₉.

### DETAILED DESCRIPTION OF THE INVENTION

In one embodiment of the present invention there is presented a compound having the structure:

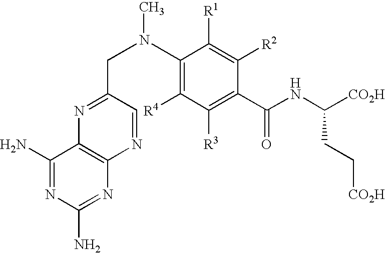

wherein R¹-R⁴ are independently fluorine or hydrogen such that at least one of and no more than two of R¹-R⁴ are fluorine. An example of this compound is N-(2-fluoro-4-amino-4-deoxy-N-methyl-pteroyl)-L-glutamic acid. In a related embodiment there is provided a pharmaceutical composition thereof and a pharmaceutically acceptable carrier.

In another embodiment of the present invention there is provided a non-invasive method of monitoring tumor tissue concentration of an anticancer drug in real time, comprising administering an amount of the compound described supra over a period of time to an individual with the tumor; selecting at least one time point during administration of the compound, after administration of the compound or a combination thereof; acquiring a magnetic resonance spectrum of a ¹⁹F chemical shift of the compound in a volume of the tumor at the time point(s); and positively correlating a ratio of signal intensities of the ¹⁹F shift and an external standard with amount of the compound in the volume of tumor at the time point(s), thereby monitoring the tumor tissue concentration of the compound in real time.

In one aspect of this embodiment the method further comprises positively correlating the concentration of the compound in the tumor tissue at the time point selected after the end of the period of administration with tumor sensitivity to the compound or with tumor resistance to the compound, where a high concentration of the compound relative to the amount administered correlates with tumor sensitivity or a low concentration of compound relative to the amount administered correlates with tumor resistance. Alternatively, in this aspect, the concentration of the compound in the tumor tissue at the time point selected near the middle of the period of administration is compared with the concentration of the compound in the tumor tissue at the time point selected near the end of the period of administration, where an increase in concentration correlates with tumor sensitivity or a decrease or no increase in concentration correlates with tumor resistance to the compound.

In these aspects an example of a time point after the end of the period of administration is at about 4 hours to about 8 hours. An example of a time point near the middle of the period of administration is at about 2 hours.

In a related aspect the method further comprises devising a therapeutic strategy to reduce tumor burden based on the sensitivity of or resistance of the tumor to the compound and reducing tumor burden of the sensitive tumor via continued administration of the compound described supra or alternatively, reducing tumor burden of the resistant tumors via administration of a different anticancer compound.

In certain aspects of this embodiment the tumor may be an osteosarcoma, a head and neck sarcoma, a bladder carcinoma, a brain tumor, a lymphoma or a leukemia. An example of a period of administration of the compound described supra is about 1 hour to about 6 hours. The amount of the compound administered may be about 100 mg/kg body weight to about 1000 mg/kg body weight. A representative amount is about 400 mg/kg body weight. Alternatively, the amount may be measured as about 1 gm/m² to about 12 g/m² body surface area.

In another embodiment of the present invention there is provided a method of non-invasively categorizing a tumor as sensitive to or resistant to methotrexate in real time comprising administering an amount of the compound described supra over a period of time to an individual with the tumor; acquiring a magnetic resonance spectrum of an ¹⁹F chemical shift of the compound in a volume of the tumor at a time point selected after the end of the period of administration; and positively correlating a ratio of signal intensities of the ¹⁹F shift and an external standard with concentration of the compound in the volume of tumor at the time point, where a high concentration of the compound relative to the amount administered indicates the tumor is sensitive to the compound or where a low concentration of the compound relative to the amount administered indicates the tumor is resistant to the compound, thereby categorizing said tumor in real time.

In an alternate embodiment the method comprises acquiring magnetic resonance spectra of an ¹⁹F chemical shift of the compound described supra in a volume of the tumor at a first time point selected near the middle of the period of administration and at a second time point selected after the end of the period of administration; and positively correlating a ratio of signal intensities of the ¹⁹F shift and an external standard with concentration of the compound in the volume of tumor at the time points; and comparing the concentrations of the compound in the tumor volume at the first and second time points, where an increase in concentration from the first time point to the second time point indicates the tumor is sensitive to the compound or, alternatively, a decrease or no increase in concentration from the first time point to the second point indicates the tumor is resistant to the compound, thereby categorizing the tumor in real time.

In aspects of both these embodiments, the method comprises devising a therapeutic strategy to reduce tumor burden as described supra. Additionally, in all aspects of these embodiments the period of administration, the time points near the middle and after the end of the period of administration, the types of tumor and the amount of the compound administered are as described supra.

In yet another embodiment of the present invention there is provided a method of treating a cancer sensitive to methotrexate in an individual comprising administering a therapeutic amount of the compound described supra over a continuous period of time at least once to the individual to reduce tumor burden of the cancer thereby treating the cancer.

In one aspect of this embodiment the method further comprises acquiring a magnetic resonance spectrum of an ¹⁹F chemical shift of the compound in a volume of the tumor at a time point selected after the end of the period of administration; and positively correlating a ratio of signal intensities of the ¹⁹F shift and an external standard with concentration of the compound in the volume of tumor at the time point to determine if the tumor is acquiring resistance to the compound, where a high concentration of the compound relative to the amount administered indicates the tumor is not acquiring resistance to the compound.

In another aspect the method further comprises acquiring magnetic resonance spectra of an ¹⁹F chemical shift of the compound in a volume of the tumor at a first time point selected near the middle of the period of administration and at a second time point selected after the end of the period of administration; positively correlating a ratio of signal intensities of the ¹⁹F shift and an external standard with concentration of the compound in the volume of tumor at the first and second time points; and comparing the concentrations of the compound in the tumor volume at the time points to determine if the tumor is acquiring resistance to the compound, where a decrease or no increase in concentration from the first time point to the second time point correlates with acquired tumor resistance to the compound.

Further to these aspects the method comprises devising an alternate therapeutic strategy if the tumor is acquiring resistance to the compound and administering a different anticancer compound to treat the resistant tumor. In all aspects of this embodiment the period of administration, the time points near the middle and after the end of the period of administration, the types of tumor and the amount of the compound administered are as described supra.

The following abbreviations are used herein. MTX: methotrexate; MR: magnetic resonance; EI: electron ionization; MS: mass spectrometry; IR: infrared; TLC: thin layer chromatography; PDB: protein data bank; RFC: reduced folate carrier; DMF: dimethylformamide; TFA: trifluoroacetic acid; DHFR: dihydrofolate reductase; XTT: sodium 3′-[1-(phenylaminocarbonyl)-3,4-tetrazolium]-bis(4-methoxy-6-nitro) benzene sulfonic acid hydrate; and PMS: phenazine methosulfate.

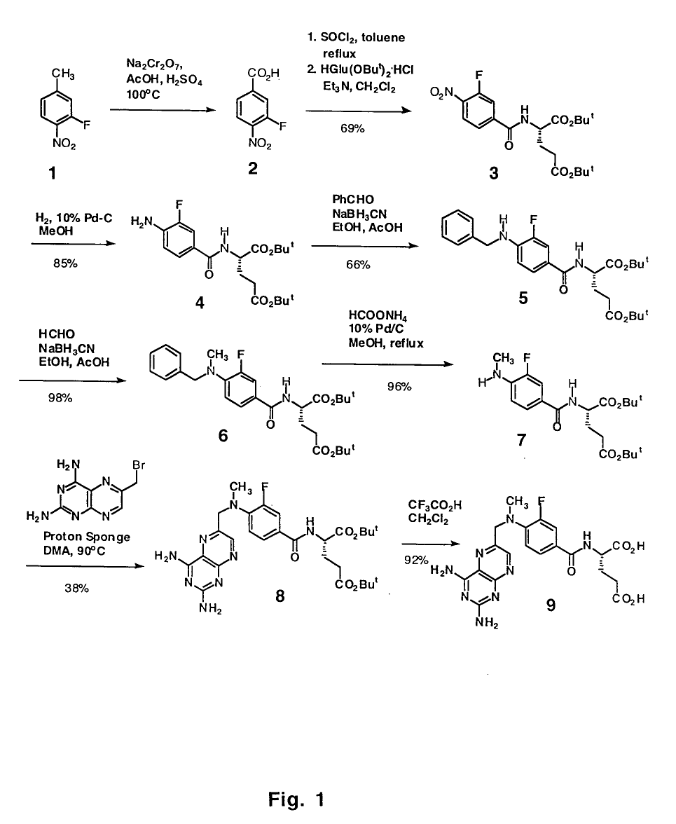

Provided herein is a novel fluorine-labeled analog of methotrexate, 3′-fluoromethotrexate having both clinical utility as a diagnostic agent and therapeutic efficacy as an anticancer drug. The synthetic route for 3′-fluoromethotrexate was essentially the same as that of 3′-fluoroaminopterin (6). The synthesis was readily accomplished in eight steps starting from 3-fluoro-4-nitro-toluene 1 as outlined in scheme A (FIG. 1).

Thus standard sodium dichromate oxidation of 3-fluoro-4-nitro-toluene 1 afforded the 3-fluoro-4-nitro-benzoic acid 2, which was subsequently converted into the acid chloride by treatment with thionyl chloride in refluxing toluene. Addition of the isolated acid chloride to di-tert-Butyl-L-glutamate hydrochloride in dichloromethane containing two equivalents of triethylamine at 0° C. gave the amide, di-tert-butyl N-(3-fluoro-4-nitrobenzoyl)-L-glutamate 3, in 69% yield. At this point the hydrogenolysis of the nitro group to the corresponding amine was done at atmospheric pressure and room temperature to give the p-aminobenzoyl moiety, di-tert-butyl N-(3-fluoro-4-aminobenzoyl)-L-glutamate, 4 in 85% yield. The resulting amine was transformed into the corresponding benzyl amine, di-tert-butyl N-(3-fluoro-4-benzylaminobenzoyl)-L-glutamate 5, by reductive amination with benzaldehyde using the standard sodium cyanoborohydride methodology.

With the benzyl group in place, monomethylation of the benzyl amine was easily achieved via a subsequent second reductive amination with formaldehyde to give methylbenzyl amine, di-tert-butyl N-(2-fluoro-4-benzylmethylaminobenzoyl)-L-glutamate 6, in 98% yield. Hydrogenolysis using 10% palladium on carbon with ammonium formate in refluxing methanol gave methyl amine, di-tert-butyl N-(2-fluoro-4-methylaminobenzoyl)-L-glutamate 7, in 90% yield. Condensation with 6-(bromomethyl)-2,4-pteridinediamine hydrobromide (20-21) in dimethylacetamide at 90° C. with proton sponge gave the fully assembled di-tert-butyl-3′-fluoromethotrexate, di-tert-butyl N-(2-fFluoro-4-amino-4-deoxy-N-methyl-pteroyl)-L glutamate 8, in 38% yield after chromatography. Subsequent deprotection with trifluoroacetic acid in dichloromethane gave N-(2-Fluoro-4-amino-4-deoxy-N-methyl-pteroyl)-L-glutamic acid or 3′-fluoromethotrexate (FMTX) 9 in 92% yield.

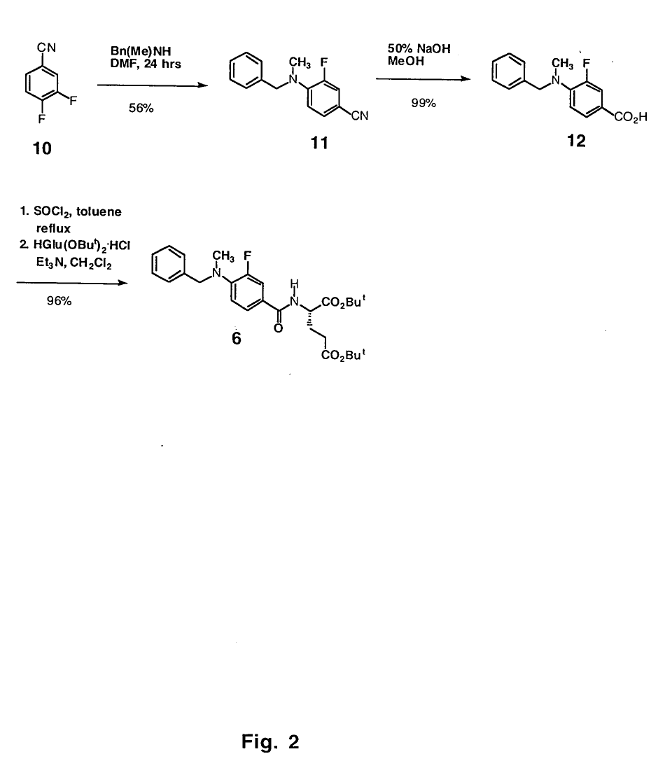

Also provided is an alternate synthetic route for compound 6 (FIG. 2). This is accomplished in three steps starting with 3,4-difluoro-benzonitrile 10. Initial displacement of the fluorine at C-4 with methyl benzylamine in DMF at room temperature afforded compound 4-(benzyl-methyl-amino)-3-fluoro-benzonitrile 11 in 98% yield. Hydrolysis of the nitrile with 50% sodium hydroxide gave the resulting acid 4-(benzyl-methyl-amino)-3-fluoro-benzoic acid 12 in 98% isolated yield. This acid was then converted to the final compound 9 as previously described in 99% yield.

Other fluorine analogs of methotrexate similar to FMTX may be synthesized using corresponding synthetic schema. Fluorine analogs, such as 2′-fluoro methotrexate, 2′3′-difluoromethotrexate, 3′5′-diflurormethotrexate or 2′6′-diflurormethotrexate, as well as N—CH₂CF₃ substitutions, may used in the diagnostic and therapeutic methods described herein. Because the methotrexate molecule is symmetrical this encompasses 4′-fluorometotrexate, 6′-fluorometotrexate and 3′,5′-difluoro methotrexate. The preparation of the 2′-fluorometotrexate would use the same scheme except 3-fluoro-4-nitro-benzoic acid would be replaced with 2-fluoro-4-nitro-benzoic acid. For the rest of the analogs mentioned above, the appropriate benzoic acid is used in the synthetic scheme. Also, fluorine-containing glutamic acid derivatives or glutamic acid mimetics may be used.

The energy-minimized structures of the complexes of MTX and FMTX with the enzymes thymidine synthetase (TS) and dihydrofolate reductase (DHFR) obtained during molecular modeling demonstrate that the introduction of the fluorine-label at the 3′ position of methotrexate results in only very small differences in the binding of the inhibitor in the antifolate-enzyme complexes. 3′-fluoromethotrexate also has biological activity and is considerably more potent in vivo than methotrexate. Therefore, the presence of the fluorine label in 3′-fluoromethotrexate causes minimal modification of the activity of the parent compound, methotrexate. Additionally, tumor uptake of 3′-fluoromethotrexate is readily detectable in vivo with good temporal resolution using 19F magnetic resonance spectroscopy.

Thus, the fluorine-labeled methotrexate provided herein may be used with ¹⁹F magnetic resonance measurements to differentiate between methotrexate sensitive and methotrexate resistant tumors and to predict therapeutic response. It is contemplated that the ability of MTX-sensitive tumors to concentrate and maintain elevated tissue levels of 3′-fluoromethotrexate for longer periods is a hallmark of therapeutic responsiveness to this antifolate. The uptake and retention of 3′-fluoromethotrexate, as evidenced by 19F MR spectroscopy, is, therefore, the diagnostic indicator.

In humans, methotrexate is routinely administered, not as a bolus, but as a 4-6 hour slow infusion, although infusion times of about 1 hour to about 6 hours may be used. For practical/financial reasons it would not be possible to routinely follow the full drug uptake time-course in tumors in the clinic. However, quantitation of the 3′-fluoromethotrexate resonance indicated that 3′-fluoromethotrexate accumulated in the tumor for about 234 minutes post-injection and remained at elevated levels until the conclusion of the ¹⁹F MR observation at 270 minutes post-injection.

Thus, the present invention provides a non-invasive diagnostic test in real time to determine sensitivity or resistance of the tumor to MTX treatment. A single late time-point ¹⁹F MR measurement of 3′-fluoromethotrexate concentration in the tissue of interest following infusion in the clinical setting can correlate to tumor sensitivity or resistance and thereby to therapeutic efficacy of MTX or 3′-fluoromethotrexate. The time point may be chosen within the range of about 0 hours to about 8 hours after the end of administration of 3′-fluoromethotrexate. A high concentration would indicate a tumor sensitive to MTX and 3′-fluoromethotrexate.

Alternatively, an earlier or mid-range time point, for example, but not limited to, about 2 hours also may be chosen during administration to compare with the later time point. An increase in concentration would be an indicator of tumor sensitivity to MTX and 3′-fluoromethotrexate. A decrease in concentration or a low level of tissue concentration at both time points may be an indicator of tumor resistance or of a tumor becoming resistant. Given what is standard in the art and what is disclosed in the instant invention, it is well within the ordinary skill of an artisan to determine the amount of 3′-fluoromethotrexate administered, to acquire the ¹⁹F MR spectra of 3′-fluoromethotrexate in the tumor volume and to correlate pharmacokinetic parameters of 3′-fluoromethotrexate with the sensitivity/resistance of the tumor to methotrexate or 3′-fluoromethotrexate.

The results of such a test may be used to direct the therapeutic strategy for each patient. For example, a newly diagnosed osteosarcoma patient might be treated with methotrexate or trimetrexate based on the results of an initial 3′-fluoromethotrexate ¹⁹F MR test. Trimetrexate is currently used in the treatment of patients with relapsed osteosarcoma because it can overcome methotrexate transport resistance, which is likely to be present in this setting. FMTX also may be used to devise therapeutic strategies for other cancers, such as, but not limited to, head and neck carcinomas, bladder carcinomas, lymphomas or leukemias.

Alternatively, the diagnostic test may be performed periodically throughout a treatment regimen to monitor tumor sensitivity to methotrexate or 3′-fluoromethotrexate. If a tumor acquires resistance or becomes significantly less sensitive, a new treatment regimen with other efficacious drugs may be instituted. Furthermore, the significant correlation between tumor concentration/time and the resulting therapeutic response may be a predictor of therapeutic efficacy.

Additionally, the present invention provides a method of treating osteosarcomas and other cancers, such as, but not limited to, head and neck carcinomas, bladder carcinomas, lymphomas or leukemias. While the in vitro cytotoxicity of 3′-fluoromethotrexate is shown to be equivalent to that of antifolate MTX, surprisingly 3′-fluoromethotrexate was significantly more potent in vivo than MTX. Thus, treatment regimens may be designed substituting 3′-fluoromethotrexate for the parent compound MTX. It is well within the skill of an artisan to determine dosage or whether a suitable dosage comprises a single administered dose or multiple administered doses.

It is specifically contemplated that pharmaceutical compositions may be prepared using 3′-fluoromethotrexate of the present invention. In such a case, the pharmaceutical composition comprises 3′-fluoromethotrexate and a pharmaceutically acceptable carrier. Such carriers are preferably non-toxic and non-therapeutic. A person having ordinary skill in this art would readily be able to determine, without undue experimentation, the appropriate dosages and routes of administration. An appropriate dosage may be a single administered dose or multiple administered doses and may depend on the subject's health, the progression or remission of the disease, the route of administration and the formulation used.

When used in vivo 3′-fluoromethotrexate is administered to the patient in therapeutically effective amounts, i.e., amounts that eliminate or reduce the tumor burden and via an appropriate route. The amount of 3′-fluoromethotrexate administered will typically be in the range of about 100 mg/kg to about 1000 mg/kg of patient weight. An example is about 400 mg/kg. Alternatively, the amount administered could be determined by grams per square meter of patient surface area. For example, about 1 gm/m² to about 12 gm/m² may be administered. The schedule will be continued to optimize effectiveness while balanced against negative effects of treatment.

It is contemplated that other chemotherapeutic drugs, if labeled with a fluorine tag, may be used in the magnetic resonance spectroscopic methods described herein. For example, such therapeutic drugs may be, but not limited to, cyclophosphamide, ifosfamide, Gleevec or some of the signaling pathway inhibitors currently available or in clinical trials.

The following examples are given for the purpose of illustrating various embodiments of the invention and are not meant to limit the present invention in any fashion.

### EXAMPLE 1

General Chemistry/molecular Modeling

High resolution ¹H NMR and ¹³C NMR spectra were recorded on a Bruker AMX-400 spectrometer using tetramethylsilane as the internal chemical shift reference standard. IR spectra were recorded on a Perkin Elmer 1600 Series Fourier transform spectrometer. Low resolution EI mass spectra were obtained on a PE SCIEX API 100 LC/MS system.

All reagents were obtained either from Sigma-Aldrich® or from Lancaster® and vacuum dried under P₂O₅ overnight before use. All solvents were reagent grade and distilled before use. Silica gel used for chromatography, MN-Kieselgel 60, was purchased from EM Science. All reactions were carried out under argon using glassware dried in an oven at 80° C. overnight and cooled under vacuum. The reaction mixtures were mechanically stirred using a magnetic stirring bar and stirring plate.

Melting points were determined using a Mel-Temp II melting point apparatus fitted with a digital Barnart 115 thermocouple thermometer, and were uncorrected. Optical rotations were recorded on a Fasco DIP-370 digital polarimeter with a sodium lamp at ambient temperature and recorded as [α]²⁰_{D} (C=g/100 ml). Molecular modeling and graphics renderings were performed using the SYBYL 6.8 (Tripos Associates Inc., St. Louis, Mo.) software package on a Silicon Graphics Octane2 R12000 workstation.

### EXAMPLE 2

Cell Lines

The human sarcoma cell lines HT-1080 (12,22), M-805 (23) and HS-16 (12,22) have been described previously. The HT-1080 cell line was obtained from ATCC (American Type Culture Collection, Rockville, Md., USA) and the M-805 and HS-16 cell lines were obtained from the laboratory of Dr. Joseph R. Bertino. The HS-16 cell line is derived from a human mesenchymal chondrosarcoma, the HT-1080, a human fibrosarcoma, and the M-805 cell line from a patient malignant fibrohistocytoma sample. Animal studies were performed according to institutionally approved protocols for the safe and humane treatment of animals.

### EXAMPLE 3

Statistics

The single factor ANOVA/Tukey test procedure (24) was performed to check for statistically significant differences between mean values, including intratumor 3′-fluoromethotrexate concentrations, area under the curve and SF, for the 3 tumor models. Significance of linear regression was verified using the F-test. Mean values are reported as mean ± sem unless otherwise indicated.

### EXAMPLE 4

Synthesis of 3′-Fluoromethotrexate

Synthesis of 3′-FMTX was achieved via two alternative synthetic schemes A and B wherein the reaction product 6 is common to both methods as detailed in FIGS. 1-2.

Scheme A: Synthesis of Intermediate 6 and FMTX

Di-tert-butyl N-(3-Fluoro-4-nitrobenzoyl)-L-glutamate 3

A stirred slurry of 3-fluoro-4-nitrobenzoic acid 2 (1 g, 5.4 mmol, 1 eq) in dry toluene (20 mL) was treated with thionyl chloride (0.6 mL, 8.10 mmol, 1.5 eq) and the mixture was heated under reflux for 2 hours. The solution was cooled to room temperature and the solvent and excess thionyl chloride were evaporated under vacuum. A solution of the resulting acid chloride in 20 mL of CH₂Cl₂ was added dropwise to a stirred mixture of di-t-butyl L-glutamate hydrochloride (1.6 g, 5.4 mmol, 1 eq) and Et₃N (1.5 mL, 10.8 mmol, 2 eq) in 20 mL of CH₂Cl₂ at 0° C. (6). The reaction mixture was allowed to warm to rt and then stirred for 2 h. The solution was washed with water, dried over Na₂SO₄ and evaporated under vacuum. The crude product was purified by column chromatography on silica gel (Hex:EtOAc, 5:1 to 4:1) to give 1.6 g (69% yield) of product 3 as a yellow syrup.

¹H NMR (CDCl₃), δ 8.12 (t, 1H, J=8.3 Hz), 7.80 (d, 1H, J=11.1 Hz), 7.74 (d, 1H, J=8.7 Hz), 7.58 (d, 1H, J=6.6 Hz, NH), 4.62 (m, 1H), 2.44-2.34 (m, 2H), 2.20 (m, 1H), 2.09 (m, 1H), 1.50 (s, 9H), 1.44 (s, 9H); ¹³C NMR (CDCl₃), δ 173.1, 170.6, 163.5 (d, J=1.3 Hz), 156.5, 153.9, 140.6 (d, J=7.2 Hz), 138.8 (d, J=7.8 Hz), 126.2 (d, J=2.5 Hz), 122.9 (d, J=4.2 Hz), 117.8, 117.5, 82.7, 81.3, 53.5, 31.5, 27.9, 27.9, 26.4; MS: [M+H]⁺ 427.1, [M+Na]⁺ 449.0, Calcd for C₂₀H₂₇N₂O₇F, 426.18.

Di-tert-butyl N-(3-Fluoro-4-aminobenzoyl)-L-glutamate 4

A solution of the nitro compound 3 (1.54 g, 3.61 mmol, 1 eq) in 25 mL of EtOAc was stirred with 10% Pd-C (134 mg) under an atmosphere of H₂ until complete reaction was indicated by TLC (1 h). The reaction mixture was filtered through Celite and evaporated under vacuum. The crude mixture was purified by column chromatography of silica (Hex:EtOAc, 2:1) to give the amino compound 4 (1.18 g, 85% yield) as a white crystal.

Melting point=83-84° C.; ¹H NMR (d₆-DMSO), δ 8.23 (d, 1H, J=7.6 Hz), 7.59 (dd, 1H, J=1.8, 12.8 Hz), 7.52 (d, 1H, J=1.8, 8.3 Hz), 6.78 (t, 1H, J=8.7 Hz), 4.34 (m, 1H), 2.33 (t, 2H, J=7.5 Hz), 2.13 (br s, 2H, NH₂), 2.02 (m, 1H), 1.92 (m, 1H), 1.39 (s, 9H), 1.24 (s, 9H); ¹³C NMR (d₆-DMSO), δ 171.5, 171.3, 150.5, 148.1, 139.8, 139.7, 124.6, 120.8 (d, J=5.3 Hz), 114.5 (d, J=4.7 Hz), 114.2, 114.0, 80.2, 79.6, 52.3, 31.3, 27.6, 27.5, 25.9; MS: [M+H]⁺ 397.0, [M+Na]⁺ 419.0, Calcd for C₂₀H₂₉N₂O₅F, 396.21.

Di-tert-butyl N-(3-Fluoro-4-benzylaminobenzoyl)-L-glutamate 5

Sodium cyanoborohydride (1.67 g, 26.54 mmol, 2.4 eq) was added in small portions to a stirred solution of 4 (4.39 g, 11.06 mmol, 1 eq), benzaldehyde (1.6 mL, 15.49 mmol, 1.4 eq) and bromocresol green (˜1 mg) in 50 mL of ethanol. After complete addition, acetic acid was added dropwise to adjust the solution to slightly acidic pH as determined by the indicator. The reaction solution was stirred for 24 h at room temperature and, during this time, acetic acid was added dropwise as necessary to maintain a slightly acidic solution. The acidic solution was extracted with EtOAc/Et₂O (1:1, v:v). The organic phase was washed with 10% NaOH, water and saturated NaCl solution, dried over Na₂SO₄ and evaporated under vacuum to afford the crude product. Purification by column chromatography on silica gel (Hex:EtOAc, 3:1 to 1:1) gave the product 5 (3.54 g, 66%) as a white solid and recovered starting material 4 (909 mg, 21%).

Melting point=80-82° C.; ¹H NMR (CDCl₃), δ 7.53 (dd, 1H, J=2.0, 12.4 Hz), 7.45 (dd, 1H, J=1.7, 8.4 Hz), 7.37 (m, 5 H), 6.78 (d, 1H, J=7.3 Hz), 6.63 (t, 1H, J=8.4 Hz), 4.63 (m, 1H), 4.42 (s, 2H), 2.39 (m, 1H), 2.32 (m, 1H), 2.20 (m, 1H), 2.02 (m, 1H), 1.59 (br s, 1H), 1.48 (s, 9H), 1.27 (s, 9H); ¹³C NMR (CDCl₃), δ 172.6, 171.4, 165.9, 151.8, 149.4, 139.6, 139.5, 138.0, 128.8, 127.5, 127.3, 124.0, 121.9 (d, J=5.7 Hz), 113.9, 113.6, 110.9(d, J=3.5 Hz), 82.3, 80.8, 52.7, 47.3, 31.6, 28.0, 27.5; MS: [M+H]⁺ 487.3, [M+Na]⁺ 509.3, Calcd for C₂₇H₃₅N₂O₅F, 486.25.

Di-tert-butyl N-(2-Fluoro-4-benzylmethylaminobenzoyl)-L-glutamate 6

Sodium cyanoborohydride (722 mg, 72.7 mmol, 1.5 eq) was added in small portions to a stirred solution of benzylamino compound 5 (3.54 g, 10.91 mmol, 1 eq) and formaldehyde (37% aqueous, 5.5 mL, 72.5 mmol, 10 eq) in 50 mL acetic acid at room temperature. The reaction solution was stirred for 1 h at room temperature. The acetic acid was evaporated under vacuum and the resulting residue was dissolved in EtOAc/Et₂O (1:1, v:v), washed with 1 N NaOH, water and saturated NaCl solution, dried over Na₂SO₄ and evaporated under vacuum to afford the crude product. Purification by column chromatography on silica gel (Hex:EtOAc, 3:1) gave the product 6 (3.57 g, 98%).

Scheme B: an Alternate 3-step Synthesis of Compound 6.

Benzylmethylamine (1.5 mL, 11.85 mmol, 5 eq) was added dropwise to a solution of 3,4-difluorobenzonitrile 10 (330 mg, 2.37 mmol, 1 eq) in dry DMF (5 mL) at 0° C. The reaction mixture was allowed to stir at room temperature overnight. The DMF was evaporated under vacuum and the residue chromatographed on silica gel (Hex:EtOAc, 9:1) to give 319 mg of pure product 4-(benzyl-methyl-amino)-3-fluoro-benzonitrile 11 (56%). This product was hydrolyzed with 50% of sodium hydroxide in methanol to reflux overnight. The reaction mixture was cooled to 0° C. and concentrated HCl was added dropwise to pH=2. The acidic solution was extracted with EtOAc. The organic phase was washed with saturated NaCl, dried over Na₂SO₄ and evaporated under vacuum to give 341 mg of the crude carboxylic acid 4-(benzyl-methyl-amino)-3-fluoro-benzoic acid 12 (99% yield). A stirred slurry of the carboxylic acid (341 mg, 1.31 mmol, 1 eq) in dry toluene (8 mL) was treated with thionyl chloride (144 μL, 1.97 mmol, 1.5 eq) and the mixture was heated under reflux for 2 hours. The solution was cooled to rt and the solvent and excess thionyl chloride were evaporated under vacuum. A solution of the resulting acid chloride in 8 mL of CH₂Cl₂ was added dropwise to a stirred mixture of di-t-butyl L-glutamate hydrochloride (388 mg, 1.31 mmol, 1 eq) and Et₃N (0.4 mL, 2.62 mmol, 2 eq) in 8 mL of CH₂Cl₂ at 0° C. The reaction mixture was allowed to warm to rt and then stirred for 2 h. The solution was washed with water, dried over Na₂SO₄ and evaporated under vacuum. The crude product was purified by column chromatography on silica gel (Hex:EtOAc, 3:1) to give 633 mg (96% yield) of product 6 as a white solid.

Melting point=71-72° C.; IR (NaCl) 3346 (br m), 2978 (s), 2933 (m), 1730 (s), 1641 (s), 1616 (s), 1537 (m), 1509 (s), 1453 (m), 1368 (s), 1255 (s), 1153 cm⁻¹ (vs); ¹H NMR (CDCl₃), δ 7.54 (dd, 1H, J=2.1, 14.3 Hz), 7.48 (dd, 1H, J=2.1, 8.4 Hz), 7.34-7.24 (m, 5H), 6.89 (d, 1H, J=7.4 Hz), 6.81 (t, 1H, J=8.7 Hz), 4.65 (dt, 1H, Jt=7.8 Hz, Jd=4.5 Hz), 4.44 (s, 2H), 2.86 (s, 3H), 2.43 (ddd, 1H, J=6.9, 8.3, 16.6 Hz), 2.31 (ddd, 1H, J=6.5, 7.9, 16.6 Hz), 2.26-2.17 (m, 1H), 2.08-1.98 (m, 1H), 1.49 (s, 9H), 1.42 (s, 9H); ¹³C NMR (CDCl₃), δ 172.7, 172.3, 166.2 (d, J=1.3 Hz), 154.8, 152.4, 142.9 (d, J=7.5 Hz), 138.3, 128.8, 128.1, 127.5, 127.4, 125.3 (d, J=6.6 Hz), 123.9, 117.6 (d, J=3.4 Hz), 116.1, 115.8, 82.5, 80.9, 58.7 (d, J=6.8 Hz), 53.1, 39.2, 32.1, 28.4, 27.6; MS: [M+H]⁺ 501.2, [M+Na]⁺ 523.2, Calcd for C₂₈H₃₇N₂O₅F, 500.27; [α]²⁰_{D}+8.1 (c 1.13, CHCl₃).

Di-tert-butyl N-(2-Fluoro-4-methylaminobenzoyl)-L-glutamate 7

Ammonium formate (1.12 g, 17.81 mmol, 5 eq) was added to a stirred suspension of benzylmethylamino compound 6 (3.57 g, 7.12 mmol, 1 eq) and 10% palladium on carbon (264 mg) in 50 mL of methanol at room temperature. The mixture was heated to reflux until TLC showed complete reaction. The reaction mixture was filtered through Celite and evaporated under vacuum. The crude mixture was purified by column chromatography of silica (Hex:EtOAc, 2:1) to give the amino compound 7 (2.82 g, 96% yield) as a white solid.

Melting point=95-96° C.; IR (NaCl) 3377 (br), 2979, 2933, 1726, 1658, 1641, 1620,1580, 1548, 1513, 1502, 1451, 1367, 1154 cm⁻¹; ¹H NMR (CDCl₃) δ 7.53-7.47 (m, 2H), 6.86 (d, 1H, J=7.5 Hz), 6.62 (t, 1H, J=8.5 Hz), 4.65 (m, 1H), 4.29 (br s, 1H, NH), 2.92 (d, 3H, J=5.2 Hz), 2.43 (m, 1H), 2.31 (m, 1H), 2.22 (m, 1H), 2.03 (m, 1H), 1.49 (s, 9H), 1.42 (s, 9H); ¹³C NMR (CDCl₃) δ 172.5, 171.5, 166.0 (d, J=2.1 Hz), 151.6, 149.3, 140.7, 140.6, 123.9, 121.2 (d, J=5.8 Hz), 113.3, 113.1, 109.8 (d, J=3.7 Hz), 82.1, 80.6, 52.6, 31.6, 29.6, 27.9, 27.87, 27.40; MS: [M+H]⁺ 411.1, [M+Na]⁺ 433.1, Calcd for C₂₁H₃₁N₂O₅F, 410.22; [α]²⁰_{D}+10.5 (c 1.48, CHCl₃).

Di-tert-butyl N-(2-Fluoro-4-amino-4-deoxy-N-methyl-pteroyl)-L glutamate 8

The amine 7 (887 mg, 2.16 mmol, 1 eq) was stirred with bromomethyldiaminopteridine which was prepared according to the method of Piper and Montgomery (20) as modified by Boyle and Pfleiderer (21) (80% purity, 1.02 g, 2.46 mmol, 1.1 eq) in 6 mL of dry DMAC at 90° C. for 1 h. “Proton sponge” (Aldrich Chemical Co., 480 mg, 2.24 mmol, 1 eq) was then added and the reaction was continued for an additional hour. The solvent was removed under vacuum. The dark residue was treated with 50 mL of CHCl₃ containing 1 mL of Et₃N and adsorbed in silica gel. Column chromatography on silica gel (CHCl₃:EtOH, 4:1) provided almost pure product. Another column chromatography was performed to obtain 477 mg of pure product 8 (38% yield) as a yellow solid.

Melting point=160-164° C.; ¹H NMR (d₆-DMSO), δ 9.09 (br s), 8.65 (s, 1H), 8.40 (d, 1H, J=7.5 Hz), 7.87 (br s), 7.67 (m, 2H), 7.56 (br s), 7.06 (t, 1H, J=8.7 Hz), 6.82 (br s), 4.62 (s, 2H), 4.29 (m, 1H), 3.00 (s, 3H), 2.31 (t, 2H, J=7.3 Hz), 2.03-1.87 (m, 2H), 1.39 (s, 9H), 1.37 (s, 9H); ¹³C NMR (d₆-DMSO), δ 171.4, 171.1, 165.1 (d, J=1.5 Hz), 162.7, 162.8, 155.0, 153.6, 151.2, 149.3, 145.5, 141.4, 141.3, 125.0 (d, J=6.4 Hz), 124.3, 121.2, 117.6 (d, J=3.6 Hz), 115.3, 115.1, 80.5, 76.7, 56.9 (d, J=6.6 Hz), 52.4, 40.3, 31.3, 27.6, 27.6, 25.1; MS: [M+H]⁺ 585.4, [M+Na]⁺ 607.4, Calcd for C₂₈H₃₇N₈O₅F, 584.28.

N-(2-Fluoro-4-amino-4-deoxy-N-methyl-pteroyl)-L-glutamic acid 9

The protected fluoromethotrexate 8 (750 mg, 1.28mmol) was dissolved in 12 mL of CH₂Cl₂ and 6 mL of TFA was added dropwise. After stirring for 2 h at rt the reaction mixture was thoroughly evaporated under vacuum. The residue was dissolved in 0.01 N NaOH. The pH was the adjusted to 4.2-4.4 by addition of 1N HCl giving a bright yellow precipitate. Filtration and washing with cold water provided the final product 9 (554 mg, 92% yield) as a bright yellow solid.

Melting point=220-223° C. (decomp.); ¹H NMR (d₆-DMSO), δ 8.65 (s, 1H), 8.44 (d, 1H, J=7.6 Hz), 7.76 (br s, 1H), 7.69 (m, 2H), 7.42 (br s, 1H), 7.06 (t, 1H, J=8.8 Hz), 6.76 (br s, 2H), 4.62 (s, 2H), 4.36 (m, 1H), 3.00 (s, 3H), 2.32 (t, 2H, J=7.5 Hz), 2.07 (m, 1H), 1.92 (m, 1H); ¹³C NMR (d₆-DMSO), δ 173.9, 173.5, 165.1, 162.7, 162.5, 154.6, 153.7, 151.3, 145.9, 141.4, 141.4, 125.2, 125.2, 121.3, 117.7, 57.0, 51.9, 40.0, 30.5, 26.0; MS: [M+H]⁺472.9, Calcd for C₂₀H₂₁N₈O₅F, 472.16; [α]²⁰_{D}+16.5 (c 0.87, 1N NaOH).

### EXAMPLE 5

Molecular Modeling

The PDB coordinate file for the thymidylate synthetase-methotrexate cocrystal structure was obtained from The Protein Data Bank (1AXW). The atom types for the inhibitor and cofactor dUMP were corrected, hydrogen atoms were added and the protein C and N endgroups were fixed using the SYBYL/BIOPOLYMER module. Protein atomic charges were assigned with the Kollman all-atom charge set and inhibitor/cofactor charges were calculated using the Gasteiger-Hückel method. The complex was minimized using the Powell method, the Tripos Force Field and 0.05 kcal/mol·Å rms gradient as the convergence criterion. All protein and cofactor heavy atoms, inherent to the crystal structure, were constrained in an aggregate during minimizations. Surfaces and cartoon diagrams were created within SYBYL using the MOLCAD surface dialog.

Since the position of the ligand atoms in the x-ray structure with a minimized model cannot be directly compared, a minimized MTX (methotrexate):TS (thymidylate synthetase) template was required. To create this model, the MTX:TS complex was minimized using the methods above. The conformation of MTX in the binding pocket changed only very slightly after minimization. MTX was replaced with fluoromethotrexate (FMTX) and was again minimized as above. An analogous study of the dihydrofolate reductase:MTX and FMTX complexes with NADPH was performed beginning with the X-ray structure of the MTX:DHFR complex of the M. tuberculosis DHFR enzyme (Li et al, 2000).

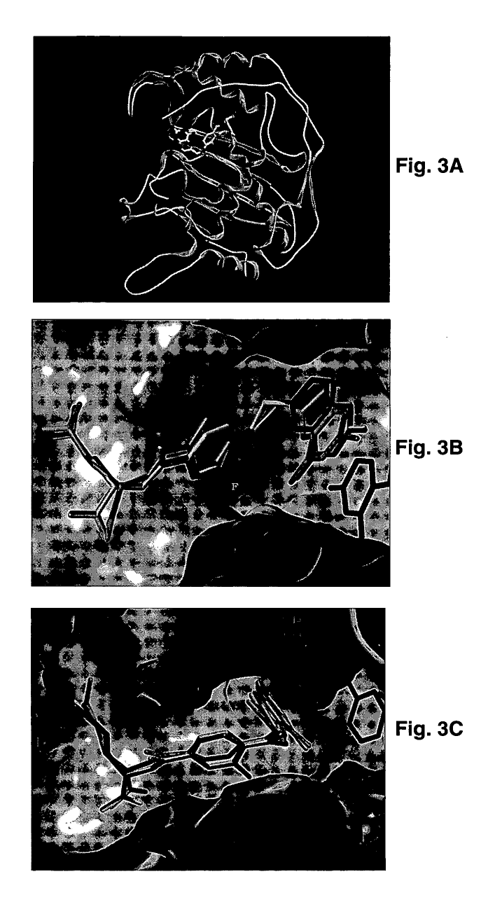

The energy-minimized structures of the complexes of MTX and FMTX with the enzymes thymidine synthetase (TS) and dihydrofolate reductase (DHFR) demonstrate that the introduction of the fluorine-label at the 3′ position of MTX results in only very small differences in the binding of the inhibitor in the antifolate-enzyme complexes. FIGS. 3A-3C show the very small differences in the geometry of the inhibitors MTX and FMTX in these energy minimized computer models. The modeling results also indicate only very slight differences in the binding energy between the MTX-enzyme and FMTX-enzyme complexes. The FMTX complexes appear to be slightly more favorable energetically, but not to a significant extent. Binding energies of the antifolate complexes with TS and DHFR differ by 1.4 and 3.7 kcal/mol, respectively. This suggests that FMTX may bind slightly better than MTX in both proteins.

### EXAMPLE 6

In Vitro Cytotoxicity Against HT-1080

The cytotoxicity of 3′FMTX was compared with that of the parent compound MTX against the methotrexate-sensitive human sarcoma cell line HT-1080 (ATCC, Rockville, Md., USA). Cultured cells were maintained as monolayer cultures in RPMI 1640 culture medium supplemented with 10% fetal calf serum at 37° C. under a humidified 5% CO₂ atmosphere.

For cytotoxicity assays monolayer cells were trypsinized and plated in 6 well culture plates (10 cm² per well) at a density of 1000 cells/well. After a period of 48 hours to allow for cell attachment and the establishment of cell proliferation the medium was aspirated and replaced with fresh medium. During drug exposure culture medium was either normal medium or thymidine-free medium in order to determine the effect of thymidine salvage (25-26).

Thymidine-free medium was prepared from normal medium via treatment with thymidine phosphorylase (Sigma, St. Louis, Mo.) for 60 min at 37° C. followed by heat inactivation of the thymidine phosphorylase at 55° C. and filtration (0.22 μm filter). MTX and FMTX stock solutions (10 mM) were prepared in isotonic saline with pH adjusted to 7.4. After a period of 24 hours drug exposure, culture medium was aspirated and replaced with fresh normal medium. Cells were incubated for a further 72-96 hours and cell viability was determined via the XTT/PMS assay (27-28).

This spectrophotometric assay indicates the level of cellular biochemical redox activity in each culture well relative to control, untreated cells as a measure of cell viability. Cytotoxicity, repeated in triplicate, was evaluated from drug concentration response curves and is reported in terms of IC₅₀, the concentration of drug that reduces HT-1080 cell viability by 50%. Results for MTX and FMTX were compared via Student's t-test.

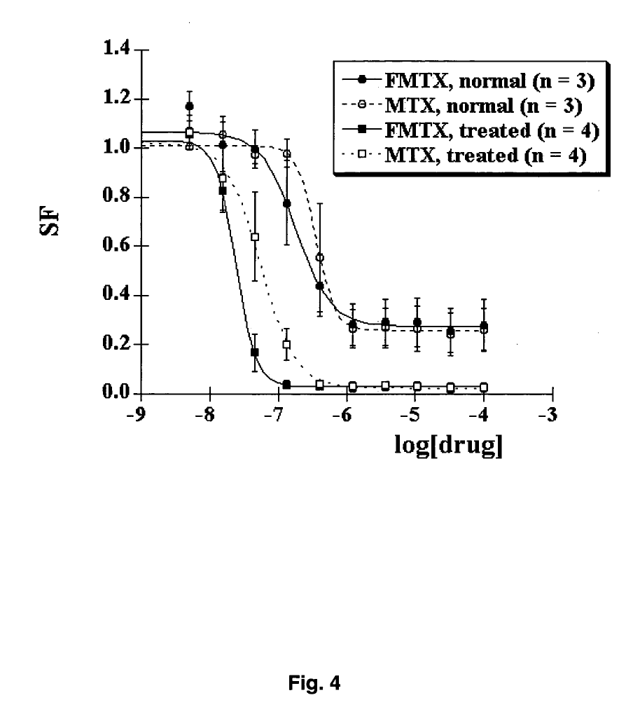

The cytotoxicity data for the two antifolates, MTX and 3′-fluoromethotrexate against the methotrexate-sensitive human sarcoma cancer HT-1080 are shown in Table 1 and in FIG. 4. Table 1 shows the IC₅₀ values for the antifolates methotrexate and 3′-fluoromethotrexate in normal RPMI-1640 culture medium and in medium without exogenous thymidine. The cytotoxicity of 3′-fluoromethotrexate is slightly greater than that of the unlabelled MTX although these differences are not statistically significant (p>0.05). These biological results are to be expected based upon the similar binding energies of MTX and 3′-fluoromethotrexate for the target enzymes DHFR and TS predicted by molecular modeling. It is also apparent that the presence of exogenous thymidine in the culture medium reduces cytotoxicity.

TABLE 1
IC₅₀ values for the antifolates MTX and FMTX

| Drug/medium | IC50 for 24 h |
|---|---|
| Condition * | drug exposure |
| FMTX/normal medium | 345 ± 117 nM |
| MTX/normal medium | 445 ± 148 nM |
| FMTX/treated medium | 26 ± 5 nM |
| MTX/treated medium | 69 ± 19 nM |

* normal = RPMI-1640 medium + 10% FCS
treated = thymidine-free RPMI-1640 + 10% FCS

### EXAMPLE 7

In vitro cytotoxicity against HT-1080, M-805 and HS-16 cell lines

The cytotoxicity of 3′-fluoromethotrexate was compared with that of the parent compound, MTX, against all 3 cell lines. FMTX was synthesized according to published methods (30), MTX was obtained from Immunex (Seattle, Wash.). Cells were maintained as monolayer cultures in RPMI-1640 culture medium supplemented with 10% fetal calf serum at 37° C. under a humidified 5% CO₂ atmosphere. For cytotoxicity assays, monolayer cells were trypsinized and plated in 6 well culture plates (10 cm²/well). After a period of 48-72 hours to allow for cell attachment and establishment of cell proliferation, the medium was aspirated and replaced with fresh medium and MTX or 3′-fluoromethotrexate. Following a 24 hour drug exposure, culture medium was aspirated and replaced with fresh culture medium. Cells were incubated for a further 72-96 hours and cell viability was determined via the XTT/PMS assay (27). Cytotoxicity was evaluated from drug concentration/response curves.

### EXAMPLE 8

In Vivo 19F MR Spectra of FMTX

An in vivo ¹⁹F MR study was performed according to institutionally approved protocols for the safe and humane treatment of animals. A human prostate cancer LNCaP tumor xenograft in a nude mouse was used to determine in vivo MR visibility of 3′-fluoromethotrexate. Tumor growth was initiated by the injection of 0.2 cc's of a slurry of ˜10⁵ cells. The tumor cell slurry was inoculated subcutaneously into the left flank of 6-week-old male nude (athymic) mice.

The ¹⁹F chemical shift of 3′-fluoromethotrexate was first determined at high field (B₀=9.4 T). FMTX dissolved in blood plasma was placed in the outer compartment of a coaxial 5 mm NMR tube with the inner compartment, having a 3 mm outer diameter, containing trifluoroacetic acid (TFA) in D₂O. The 19FMTX resonance was observed at 46.4 ppm with respect to the TFA chemical shift reference.

FMTX was administered via i.v. bolus tail-vein injection at a dosage of 400 mg/kg, a dosage comparable to that used clinically in humans (19,30). The mouse was unanesthetized for ¹⁹F MR experiments. The mice crawl into a 60 cc syringe barrel with air holes which is used as an animal holder with the tumor protruding through a hole into a home-built 2 turn ¹⁹F MR surface coil. The inner diameter of the surface coil was 0.8 cm with tumor volume that offered maximum filling factor, i.e., 200-350 mm³.

The in vivo ¹⁹F MR study was performed at 188 MHz (B₀=4.7 T) in a wide-bore, 33 cm diameter, small animal imaging system (GE Omega) with the tumor in a temperature control/susceptibility matching water bath (31). For the in vivo ¹⁹F MR experiment (pulse and acquire) acquisition parameters included a 60° pulse-width with a pulse repetition time (T_{R}) of 2 sec, 10,000 Hz sweep-width, 1,024 complex data points per free induction decay (FID) and a summation of 256 FID's per spectrum (9 min/spectrum). An external chemical shift reference standard of 100 mM trifluoroacetic acid in D₂O was held in an 18 μL glass microsphere.

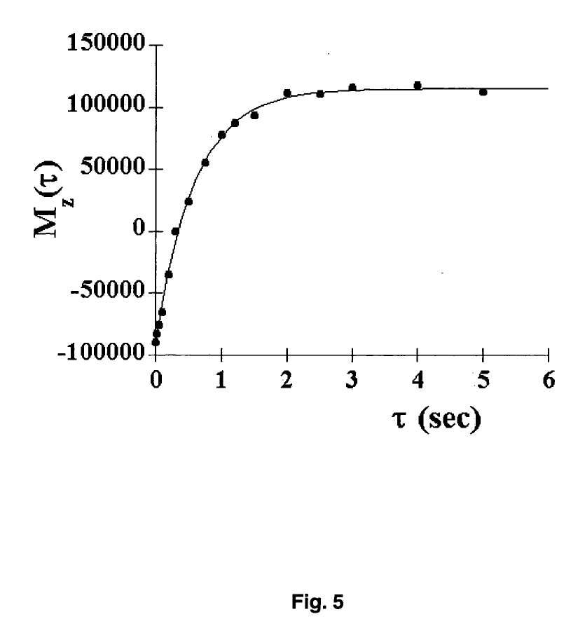

In order to optimize the acquisition parameters for future studies, the spin-lattice relaxation time T₁ was determined (32). An estimate of the in vivo FMTX T₁ value was made for a phantom sample of 7 mM FMTX dissolved in blood plasma at 37° C. and 4.7 T with the inversion-recovery pulse sequence. The resonance intensity data of the 180°-τ-90°-acquire pulse sequence was fit, as shown in FIG. 5, to the equation:

M(τ)=M₀−C·M₀·e^{-τ/T}^{₁} Eqn. (1)

wherein the constant C allows for any error in the calibration of the 90° or 180° pulses. Resonance intensities were determined via modeling of the time-domain NMR signal data using the jMRUI software package run on a PC (33). From the fit to Equation 1, a T₁ value of 0.60 sec was determined.

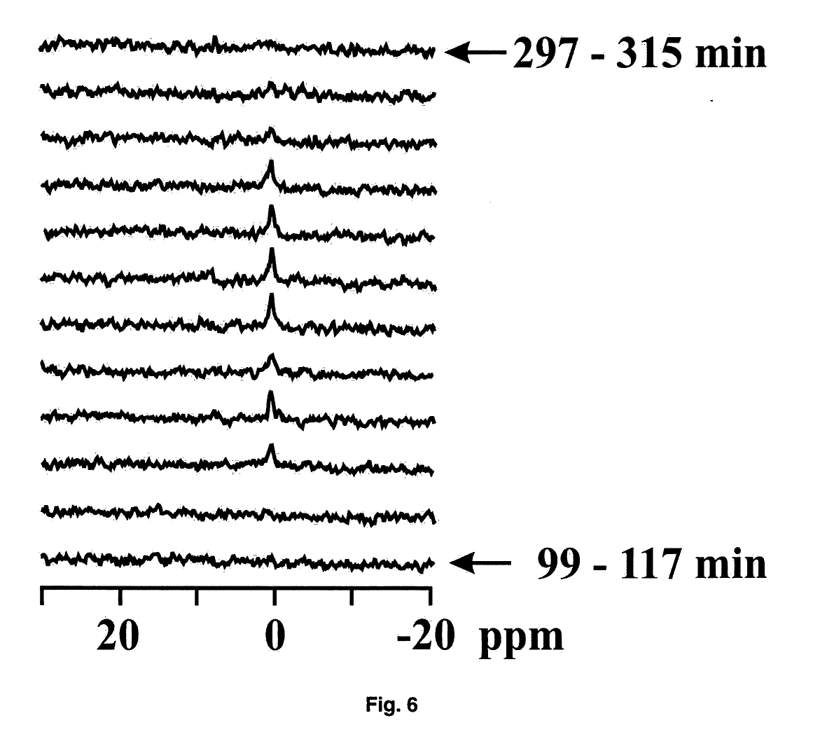

FIG. 6 demonstrates that tumor uptake of 3′-fluoromethotrexate is readily detectable in vivo with good temporal resolution using 9 minutes per spectrum. The surface coil MR experiment localizes ¹⁹F signal to the sensitive volume of the coil (i.e., the volume filled with tumor), thus it is assured that the signal intensity represents concentration of 3′-fluoromethotrexate in the tumor. In vivo the chemical shift of 3′-fluoromethotrexate is in the range from 46.5 to 46.9 ppm with respect to the external reference solution of trifluoroacetic acid. In this pilot study we did not follow the full time-course of 3′-fluoromethotrexate tumor wash-in and washout as the goal was simply to demonstrate MR visibility of the compound in vivo. Also, this set of spectra was acquired under conditions wherein longitudinal magnetization was essentially fully relaxed. Knowledge of the T₁ of FMTX in plasma (0.60 sec, as determined here) provides a good first estimate of the in vivo T₁ value at 4.7 T.

### EXAMPLE 9

Plasma Pharmacokinetics

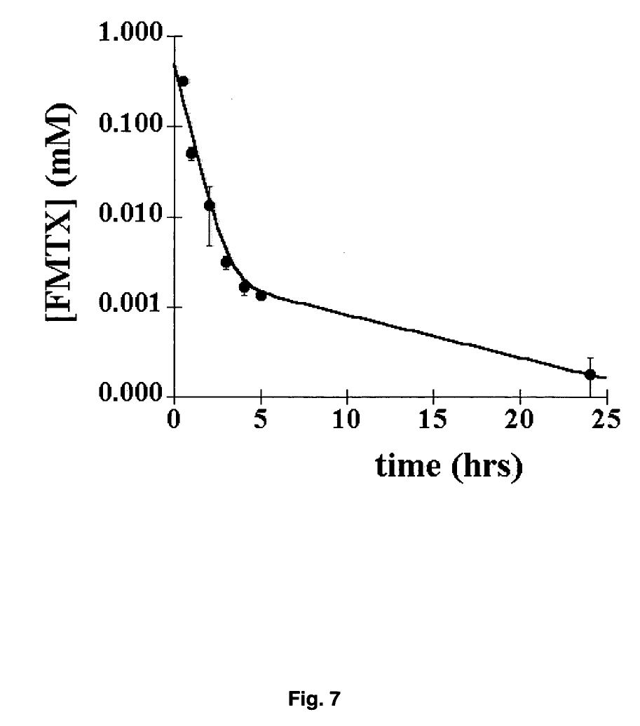

The time course of plasma pharmacokinetics was studied in mice. Each mouse received a 400 mg/kg i.v. bolus injection of 3′-fluoromethotrexate. Plasma levels of 3′-fluoromethotrexate were determined for the timepoints 0.5, 1, 2, 3, 4, 5 and 24 hr post-injection (n=3-4 per timepoint) (FIG. 7). Ten minutes prior to blood collection mice were anesthetized with ketamine/xylazine. Blood was collected via cardiac puncture into heparanized vials and centrifuged. Typical collection volumes were 0.5-1 cc and hence this was a terminal procedure.

To prepare the samples for HPLC analysis 20 μL of 50% trichloroacetic acid was added to 200 μL of plasma. Samples were allowed to stand on ice for several hours after which the precipitate was removed by centrifugation and the supernatant collected for HPLC analysis without further modification. An Agilent 1100 HPLC system (Agilent, Palo Alto, Calif.) was used. Twenty microliters of the supernatant was injected onto an Econosphere C18 5 μm 4.6×250 mm column (Econosphere, Deerfield, Ill.) with an eluent of 15% acetonitrile/85% 50 mM potassium dihydrogen phosphate at a flow rate of 1 mL/min and monitored at 313 nm. A standard curve of spiked plasma was linear from 0.31 to 10 μg/mL. Samples with higher drug concentrations were diluted to bring them to within the analytical range.

In nude mice, plasma levels of FMTX follow biexponential decay kinetics after an i.v. bolus injection of a 400 mg/kg dosage (FIG. 7). The data were fit to equation 2:

[FMTX]ₚₗₐₛₘₐ=A·e^{-k}^{₁}^{·t}+B·e^{-k}^{₂}^{·t} Eqn. (2)

The resulting fit parameters were, A=0.480 mM with rate-constant, k₁=1.74 hr⁻¹ (t_{1/2}=0.399 hr) for the fast-washout component and B=2.47×10⁻³ mM and rate-constant, k₂=0.11 hr⁻¹ (t_{1/2}=6.3 hr) for the second, slow-washout component (R=0.91). The fit results in an estimate of [FMTX]ₚₗₐₛₘₐ at t=0 min of 0.482 mM, whereas the value at the first measurement time-point, 30 minutes post-injection was 0.32±0.02 mM. By 4 hours post-injection, plasma concentrations of FMTX fell to 1.7±0.3 μM and at 24 hours decreased further to 0.18±0.09 μM.

### EXAMPLE 10

In Vivo 19F MR-observed Tumor Pharmacokinetics

Tumor xenografts were initiated by the injection of 0.1 mL's of a slurry of ˜10⁶ cells. The tumor cell slurry was inoculated subcutaneously into the left flanks of 6-week-old male athymic nude mice (Charles River, Boston, Mass.). Tumors used in this study ranged in size between 0.16 and 0.41 cc.

FMTX was administered via i.v. bolus tail-vein injection at a dosage of 400 mg/kg, a level comparable to, but less than the highest doses used clinically in humans (16,27,34). Mice were unanesthetized for 19F MR experiments. The mice crawl into a 60 cc syringe barrel with air holes which is used as an animal holder with the tumor protruding through a hole into a home-built 2 turn ¹⁹F MR solenoid coil. In vivo 19F MR studies at 188 MHz (B₀=4.7 T) were performed in a wide-bore, 33 cm diameter, small animal imaging system (Omega-Bruker, Bellerica, Mass.) with the use of a temperature control/susceptibility matching water bath (31).

For ¹⁹F MR experiments (pulse and acquire), acquisition parameters included a 60° pulse-width, a pulse repetition (T_{R}) of 0.49 sec with 2,048 transients/spectrum. Thus the temporal resolution was 18 minutes/spectrum. MR time-domain data was analyzed using jMRUI software (33). The 3′-fluoromethotrexate resonance intensity was determined relative to that of an external ¹⁹F reference standard, an 18 μL glass microsphere of 0.1 M trifluoroacetic acid in D₂O. Intratumor FMTX concentrations were estimated from the 19F MR intensity ratios using the measured longitudinal relaxation times (T₁) for TFA (3.37 s) and 3′-fluoromethotrexate (29) at 4.7 T and 37° C., the pulse angle applied to the tumor and external reference, and the tumor volume according to the method of Murphy-Boesch (35). A uniform excitation of the tumor volume was assumed. Tumor tissue concentrations of 3′-fluoromethotrexate are reported in millimoles/liter.

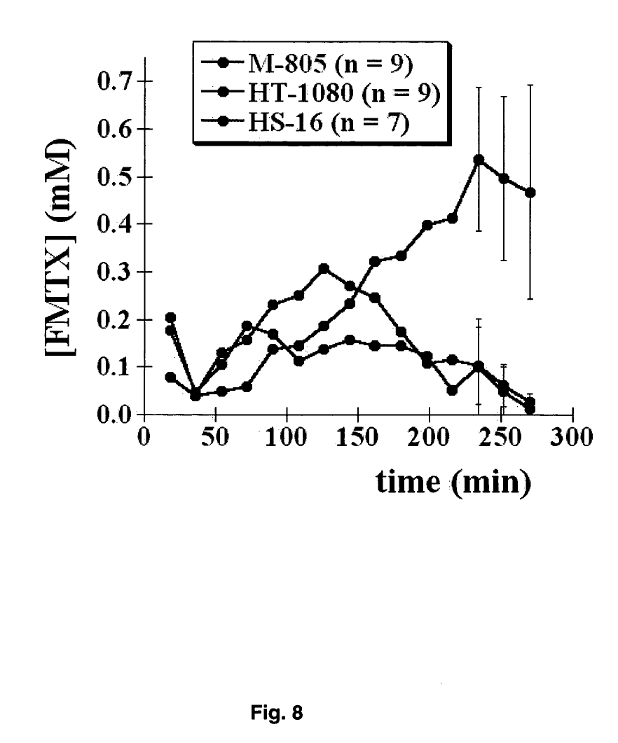

In vivo tumor tissue pharmacokinetic data acquired by ¹⁹F MR show variation in drug uptake/retention between the xenograft models investigated in this study (FIG. 8). The three tumor models (HT-1080, HS-16 and M-805) show differences both in the peak concentrations of tumor 3′-fluoromethotrexate achieved and the dynamics of uptake/retention. The MTX-sensitive HT-1080 tumor model achieves peak tissue concentrations at 234 minutes post-injection (0.54±0.15 mM), whereas in the other two models, peak tumor concentrations are lower in magnitude and are achieved at earlier times preceding washout from the tumor tissue.

In the M-805 tumor model, in which reduced uptake secondary to decreased RFC expression would be expected, peak tissue concentrations of 3′-fluoromethotrexate occurs early at 72 minutes post-injection and are considerably lower (0.19±0.08 mM) than the maximum levels observed in the HS-16 and HT-1080 tumor models. The HS-16 tumor demonstrates an ability to accumulate 3′-fluoromethotrexate in the tumor tissue, but these levels peak at 126 minutes post-administration (0.31±0.15 mM) and are followed by drug efflux from the tumor as 3′-fluoromethotrexate clears from the plasma, which is consistent with the FPGS-deficient status of this cell line. Although the mean maximum intratumor 3′-fluoromethotrexate concentrations are greatest in the HT-1080 tumor, comparison of the maximum concentrations of HT-1080 at 234 min, of M-805 at 72 min and of HS-16 at 126 min does not reveal any statistically significant differences between MTX-sensitive and resistant xenografts.

The differences in intratumor 3′-fluoromethotrexate concentrations in sensitive HT-1080 xenografts vs. resistant M-805 and HS-16 xenografts are most pronounced at later time-points. Comparison of intratumor 3′-fluoromethotrexate concentrations for the time-point centered at 234 minutes post-injection, where the 19F MR spectra is acquired over the interval 225-243 min post-injection, indicate statistically significant differences between resistant and sensitive tumor models. Intratumor 19F-MR observable concentrations at this time-point were 0.54±0.15 mM, 0.10±0.08 mM and 0.10±0.05 mM for the HT-1080, HS-16 and M-805 tumor xenograft models, respectively. These concentrations are significantly higher for the HT-1080 than the M-805 (p<0.001) and HS-16 (p<0.001) tumors, while these last two groups did not differ from each other.

Qualitatively, the shapes of the pharmacokinetic curves of model tumor FMTX uptake/retention can be understood in terms of the molecular mechanism of resistance. In the M-805 tumor model, decreased RFC activity leads to reduced tissue uptake, while in the HS-16 tumor, drug uptake is rapid with high tissue concentrations achieved, but followed by rapid egress of 3′-fluoromethotrexate from the tumor due to decreased FPGS-activity.

### EXAMPLE 11

Leucovorin Rescue and Tumor Response

At 6 hours post 3′-fluoromethotrexate administration mice received i.p. hydration of 1.0 cc normal saline and at 24 hours received leucovorin (Bedford Laboratories, Bedford, Ohio) rescue therapy (36). This protocol allows the mice to survive what would otherwise be a fatal dosage of 3′-fluoromethotrexate and is analogous to that used in high-dose MTX therapy in humans. Tumor growth was monitored post-therapy.

The 3 perpendicular axes of the tumor were measured with a micrometer and the tumor volume modeled as a spheroid, i.e., v=π/6a·b·c, where a, b, c are the dimensions of the tumor axes in cm. Tumor growth curves post-therapy were Gompertzian and hence in vivo surviving fraction, i.e., the fraction of cancer cells in the pre-treatment tumor that survived therapy SF, is reported based on the tumor volume growth parameters:

$$SF = {( \frac{1}{2} )}^{TGD / {DT}_{o}} \qquad Eqn . ( 3 )$$

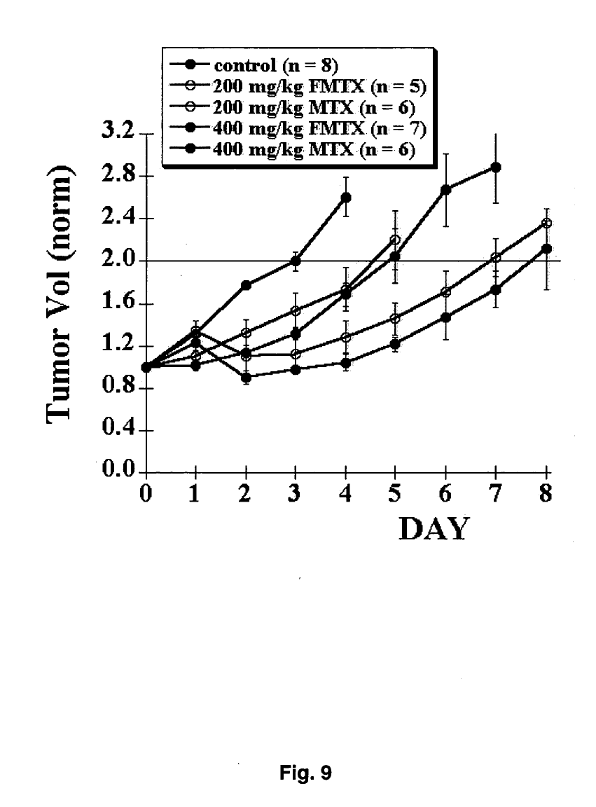

The parameter DT₀ is the mean doubling time of the untreated, control group for each xenograft tumor model and TGD is the tumor growth delay for the treated tumor (the difference in tumor doubling time between the treated tumor and DT₀). An assumption inherent in this model for determining SF is that the tumor growth rate is the same in the untreated tumor as in the post-treatment tumor during the regrowth phase, which is consistent with the observed tumor growth curves (FIG. 9). For untreated growth the tumor volume doubling times DT₀ were 2.9±0.1 days, 25.8±5.5 days and 23.4±1.9 days for the HT-1080 (n=8), HS-16 (n=4) and M-805 (n=6), respectively.

### EXAMPLE 12

FMTX vs MTX in an in Vivo MTX Sensitive Tumor Xenograft

Tumor growth curves indicate that 3′-fluoromethotrexate is considerably more potent than MTX in vivo against the MTX-sensitive HT-1080 tumor xenograft model (FIG. 9). This is somewhat surprising because in vitro, both agents have equivalent cytotoxic action against both sensitive and resistant cell lines (data not shown). Analysis of the tumor growth post-therapy allows for a comparison of the efficacy of MTX and 3′-fluoromethotrexate in terms of SF, the fraction of cells in the pretreatment tumor surviving therapy (Eqn. 3).

The volume doubling time for the HT-1080 tumor is 2.9±0.1 days (n=8). Following a 400 mg/kg i.v. bolus (FMTX or MTX), in the HT-1080 model, SF_{FMTX}=0.29±0.06 (n=7), while SF_{MTX}=0.56±0.07 (n=6), a statistically significant difference (p=0.011). Even a dosage of 200 mg/kg 3′-fluoromethotrexate was more potent than a 400 mg/kg MTX dosage, but not significantly (p=0.051). At the 200 mg/kg 3′-fluoromethotrexate dosage-level, SF=0.42±0.04 (n=5), while for 200 mg/kg methotrexate it was 0.64±0.08 (n=6).

A surprising finding is the increased efficacy of 3′-fluoromethotrexate, as compared to MTX, against the MTX-sensitive HT-1080 tumor xenograft in vivo for the 400 mg/kg dosage. Molecular modeling indicates only slightly more favorable binding of 3′-fluoromethotrexate in the active site of two of the key target enzymes, dihydrofolate reductase and thymidine synthetase (29). In vitro the two agents are equipotent against the three cell lines investigated in this study. Although not limited to such theory, it is contemplated that the distinction in vivo could occur as a result of differences in the rate of production of the inactive 7-hydroxy metabolite in the liver.

### EXAMPLE 13

Correlating Pharmacokinetic Parameters with Therapeutic Efficacy

The tumor ¹⁹F MR kinetic data suggests the use of late time-point intratumor 3′-fluoromethotrexate concentrations as a means of differentiating between sensitive and resistant tumors. The area under the curve (AUC) was estimated for the period from 225 to 279 minutes post-injection using the 19F MR intratumor concentration data (FIG. 8) and the trapezoidal rule. The calculated value is denoted as AUC₂₂₅₋₂₇₉ (in mM·min). This quantity is significantly higher for the MTX-sensitive HT-1080 at 26.4±5.7 than for either of the two resistant tumor models (p<0.05). For the HS-16 and M-805 models the values are 3.5±2.4 and 3.0±1.4, respectively, and are not significantly different.

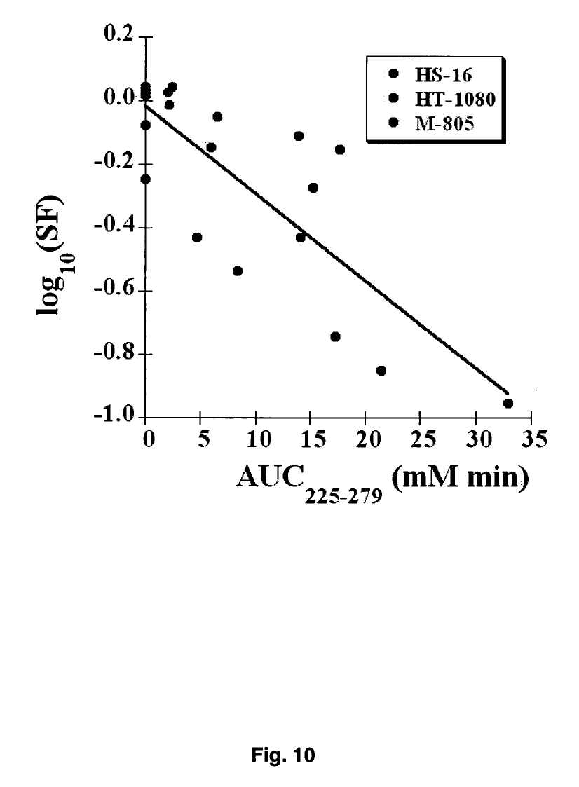

Plotting AUC₂₂₅₋₂₇₉ vs. log₁₀(SF) yields a statistically significant linear correlation across the three tumor models investigated (FIG. 10). The resulting linear least squares fit to the full data set (n=19) is:

log₁₀(SF)=−0.015−0.028·AUC₂₂₅₋₂₇₉, Eqn. (4)

(R=0.81, F=9.27, p<0.001).

This fit very nearly passes through the origin, i.e., SF=1 for AUC₂₂₅₋₂₇₉=0 mM·min).

The following references are cited herein:

1. Rudin et al., NMR in Biomedicine, 12: 69-97 (1999).

2. Artemov et al., Magn Reson Med, 34: 338-342 (1995).

3. He et al., Magn Reson Med, 33: 414-416 (1995).

4. Payne et al., Magn Reson Med, 44: 180-184 (2000).

5. Wolf W, Valuch V, Presant C A. NMR in Biomedicine, 11: 380-387 (1998).

6. Henkin J, Washtien W L. J Med Chem 26: 1193-1196 (1983).

7. Galivan et al., Proc Natl Acad Sci USA, 82: 2598-2602 (1085).

8. Yang, R. et al. Clin. Cancer Res. 9, 837-844 (2003).

9. Guo, W. et al. Clin. Cancer Res. 5, 621-627 (1999).

10. Gorlick et al., New England J Med, 335: 1041-1048 (1996).

11. Gorlick et al., Blood, 89: 1013-1018 (1997).

12. Li, W. W. et al. Cancer Res. 52, 3908-3913 (1992).

13. Hakala, M. T. Biochim. et Biophys. Acta 102, 210-225 (1965).

14. Chen, Z. S. et al. Cancer Res. 63, 4048-4054 (2003).

15. Pignon, T. et al. Cancer Chemother. Pharmacol. 33, 420-424 (1994).

16. Rousseau, A. et al. Clin. Pharmacokinet. 41, 1095-1104 (2002).

17. Nelson, S. J. Molec. Cancer Ther. 2, 497-507 (2003).

18. Salibi, N. & Brown, M. A. Clinical MR Spectroscopy: First Principles 1^{st} edn. (Wiley-Liss, NY, 1998).

19. Ackerman, et al., Nature 283, 167-170 (1980).

20. Piper et al., J Org Chem, 42: 208-211 (1977).

21. Boyle et al., Chem Ber, 113: 1514-1523 (1980).

22. Li, et al., Int. J. Cancer 68, 514-419 (1996).

23. Yang R et al. Int J. Cancer (submitted).

24. Zar, J. H. Biostatistical Analysis 2^{nd} edn. (Prentice Hall, Inc., NJ, (1984).

25. Jackman et al., Biochem Pharmacol, 33: 3269-3275, 1984.

26. O'Dwyer et al., Cancer Res, 47: 3911-3919, 1987.

27. Lamontagne et al., Cellular Pharmacol, 1: 171-174, 1994.

28. Scudiero et al., Cancer Res, 48: 4827-4833, 1988.

29. Spees, W M. et al. Molec. Cancer Ther. 2, 933-939 (2003).

30. Roberts et al., Cancer, 44: 881-890, 1979.

31. Ballon et al., Magn Reson Med, 30: 754-758, 1993.

32. Becker et al., Anal Chem, 51: 1413-1420, 1979.

33. Naressi et al., Magn Reson Materials in Phys Biol Med, 12: 141-152, 2001.

34. Zaharko, et al., J. Natl. Cancer Inst. 46, 775-784 (1971).

35. Murphy-Boesch, et al., Magn. Reson. Med. 39, 429-438 (1998).

36. Sirotnak, et al., Cancer Res. 53, 587-591 (1993).

37. Chu, E. & Takimoto, C. H. In Cancer: Principles and Practice of Oncology Vol. 1 4^{th} (ed. Devita, D. T., Rosenberg, S. A. & Hellman, S.) 358-362 (Lippincott, Williams & Wilkins, Philadelphia, Pa., 1993).

38. Li et al., J. Mol Biol, 295: 307-323, 2000.

39. Treon, et al., Clin. Chem. 42, 1322-1329 (1996).

Any publications mentioned in this specification are indicative of the levels of those skilled in the art to which the invention pertains. Further, these publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A compound having the structure 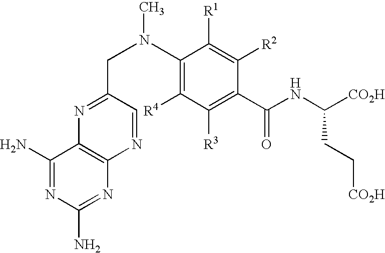
wherein R¹-R⁴ are independently fluorine or hydrogen such that at least one of and no more than two of R¹-R⁴ are fluorine.

2. The compound of claim 1, wherein said compound is N-(2-fluoro-4-amino-4-deoxy-N-methyl-pteroyl)-L-glutamic acid.

3. A pharmaceutical composition comprising the compound of claim 1 and a pharmaceutically effective carrier.

4. A non-invasive method of monitoring tumor tissue concentration of an anticancer drug in real time, comprising:
administering an amount of the compound of claim 1 over a period of time to an individual with the tumor;
selecting at least one time point during administration of the compound, after administration of the compound or a combination thereof;
acquiring a magnetic resonance spectrum of an ¹⁹F chemical shift of the compound in a volume of the tumor at said time point(s); and
positively correlating a ratio of signal intensities of said ¹⁹F shift and an external standard with amount of said compound in the volume of tumor at said time point(s) thereby monitoring the tumor tissue concentration of said compound in real time.

5. The method of claim 4, further comprising:
positively correlating the tumor tissue concentration of said compound at the time point selected after said period of administration with tumor sensitivity to said compound or with tumor resistance to said compound, wherein a high concentration of said compound relative to the amount administered correlates with tumor sensitivity or wherein a low concentration of compound relative to the amount administered correlates with tumor resistance; or
comparing the tissue concentration of said compound at the time point selected near the middle of said period of administration with the tumor tissue concentration of said compound at the time point selected after said period of administration, wherein an increase in concentration correlates with tumor sensitivity or a decrease or no increase in concentration correlates with tumor resistance to said compound.

6. The method of claim 5, wherein the time point after said period of administration is at about 0 hours to about 8 hours.

7. The method of claim 5, wherein the time point near the middle of said period of administration is at about 2 hours.

8. The method of claim 5, further comprising:
devising a therapeutic strategy to reduce tumor burden based on the sensitivity of or resistance of the tumor to said compound; and
reducing tumor burden of said sensitive tumor via continued administration of said compound; or
reducing tumor burden of said resistant tumors via administration of a different anticancer compound.

9. The method of claim 1, wherein said tumor is an osteosarcoma, a head and neck carcinoma, a bladder carcinoma, a brain tumor, a lymphoma or a leukemia.

10. The method of claim 1, wherein said administration is for a period of time of about 1 hour to about 6 hours.

11. The method of claim 1, wherein the amount of said compound administered is about 100 mg/kg body weight to about 1000 mg/kg body weight or about 1 g/m2 body surface area to about 12 g/m2 body surface area.

12. The method of claim 11, wherein the amount of said compound administered is about 400 mg/kg body weight.

13. A method of non-invasively categorizing a tumor as sensitive or resistant to methotrexate in real time, comprising:
administering an amount of the compound of claim 1 over a period of time to an individual with the tumor;
acquiring a magnetic resonance spectrum of an ¹⁹F chemical shift of said compound in a volume of the tumor at a time point selected after the end of said period of administration; and
positively correlating a ratio of signal intensities of said ¹⁹F shift and an external standard with concentration of said compound in the volume of tumor at said time point, wherein a high concentration of said compound relative to the amount administered indicates the tumor is sensitive to said compound or wherein a low concentration of said compound relative to the amount administered indicates the tumor is resistant to said compound, thereby categorizing said tumor in real time.

14. The method of claim 13, further comprising:
devising a therapeutic strategy to reduce tumor burden based on the sensitivity of or resistance of the tumor to said compound; and
reducing tumor burden of said sensitive tumor via continued administration of said compound; or
reducing tumor burden of said resistant tumor via administration of a different anticancer compound.

15. The method of claim 13, wherein said administration is for a period of time of about 1 hour to about 6 hours.

16. The method of claim 13, wherein the time point after said period of administration is at about 0 hours to about 8 hours.

17. The method of claim 13, wherein said tumor is an osteosarcoma, a head and neck carcinoma, a bladder carcinoma, a brain tumor, a lymphoma or a leukemia.

18. The method of claim 13, wherein the amount of said compound administered is about 100 mg/kg body weight to about 1000 mg/kg body weight or about 1 g/m2 body surface area to about 12 g/m2 body surface area.

19. The method of claim 18, wherein the amount of said compound administered is about 400 mg/kg body weight.

20. A method of non-invasively categorizing a tumor as sensitive or resistant to methotrexate in real time, comprising:
administering an amount of the compound of claim 1 over a period of time to an individual with the tumor;
acquiring magnetic resonance spectra of an ¹⁹F chemical shift of said compound in a volume of the tumor at a first time point selected near the middle of said period of administration and at a second time point selected after the end of said period of administration; and
positively correlating a ratio of signal intensities of said ¹⁹F shift and an external standard with concentration of said compound in the volume of tumor at said first and second time points; and
comparing the concentrations of said compound in the tumor volume at said first and second time points, wherein an increase in concentration from said first time point to said second time point indicates said tumor is sensitive to said compound or wherein a decrease or no increase in concentration from said first time point to said second time point indicates said tumor is resistant to said compound, thereby categorizing said tumor in real time.

21. The method of claim 20, further comprising:
devising a therapeutic strategy to reduce tumor burden based on the sensitivity of or resistance of the tumor to said compound; and
reducing tumor burden of said sensitive tumor via continued administration of said compound; or
reducing tumor burden of said resistant tumor via administration of a different anticancer compound.

22. The method of claim 20, wherein said administration is for a period of time of about 1 hour to about 6 hours.

23. The method of claim 20, wherein the time point after said period of administration is at about 0 hours to about 8 hours.

24. The method of claim 20, wherein the time point near the middle of said period of administration is at about 2 hours.

25. The method of claim 20, wherein said tumor is an osteosarcoma, a head and neck carcinoma, a bladder carcinoma, a brain tumor, a lymphoma or a leukemia.

26. The method of claim 20, wherein the amount of said compound administered is about 100 mg/kg body weight to about 1000 mg/kg body weight or about 1 g/m2 body surface area to about 12 g/m2 body surface area.

27. The method of claim 26, wherein the amount of said compound administered is about 400 mg/kg body weight.

28. A method of treating a cancer sensitive to methotrexate in an individual, comprising:
administering a therapeutic amount of the compound of claim 1 over a continuous period of time at least once to said individual to reduce tumor burden of said cancer thereby treating said cancer.

29. The method of claim 28, further comprising:
acquiring a magnetic resonance spectrum of an ¹⁹F chemical shift of said compound in a volume of the tumor at a time point selected after the end of said period of administration; and
positively correlating a ratio of signal intensities of said ¹⁹F shift and an external standard with concentration of said compound in the volume of tumor at said time point to determine if the tumor is acquiring resistance to said compound, wherein a high concentration of said compound relative to the amount administered indicates the tumor is not acquiring resistance to said compound.

30. The method of claim 29, further comprising:
devising an alternate therapeutic strategy if the tumor is acquiring resistance to said compound; and
administering a different anticancer compound to treat said resistant tumor.

31. The method of claim 29, wherein the time point after the end of said period of administration is at about 4 hours to about 8 hours.

32. The method of claim 28, further comprising:
acquiring magnetic resonance spectra of an ¹⁹F chemical shift of said compound in a volume of the tumor at a first time point selected near the middle of said period of administration and at a second time point selected after the end of said period of administration; and
positively correlating a ratio of signal intensities of said ¹⁹F shift and an external standard with concentration of said compound in the volume of tumor at said first and second time points; and
comparing the concentrations of said compound in the tumor volume at said first and second time points to determine if the tumor is acquiring resistance to said compound, wherein a decrease or no increase in concentration from said first time point to said second time point correlates with acquired tumor resistance to said compound.

33. The method of claim 32, further comprising:
devising an alternate therapeutic strategy if the tumor is acquiring resistant to said compound; and
administering a different anticancer compound to treat said resistant tumor.

34. The method of claim 32, wherein the time point after the end of said period of administration is at about 0 hours to about 8 hours.

35. The method of claim 32, wherein the time point near the middle of said period of administration is at about 2 hours.

36. The method of claim 29, wherein said continuous period of time is about 1 hour to about 6 hours.

37. The method of claim 28, wherein said tumor is an osteosarcoma, a head and neck carcinoma, a bladder tumor, a brain tumor, a lymphoma or a leukemia.

38. The method of claim 28, wherein the amount of said compound administered is about 100 mg/kg body weight to about 1000 mg/kg body weight or about 1 g/m2 body surface area to about 12 g/m2 body surface area.

39. The method of claim 38, wherein the amount of said compound administered is about 400 mg/kg body weight.
